(19) United States
(12) Patent Application Publication
(10) Pub. No.: US 2005/0186108 A1
(43) Pub. Date: Aug. 25, 2005

(54) Bio-air sterilization system

(76) Inventor: William Michael Fields, Highlands Ranch, CO (US)

Correspondence Address: PATTON BOGGS, 1660 LINCOLN ST, SUITE 2050, DENVER, CO, 80264, US

(21) Appl. No.: 10/783,665

(22) Filed: Feb. 20, 2004

(51) Int. Cl. A61L 9/20; A61L 9/22

(52) U.S. Cl. 422/4; 422/121; 422/123; 422/122; 422/24

(57) ABSTRACT

The bio-air sterilization system removes harmful contaminants and particulates, such as bacteria, viruses and molds, from air within an enclosed area, as well as from the surfaces located within the enclosed area, while also maintaining a safe workplace for individuals. The bio-air sterilization system uses a compressor to circulate indoor air through a pre-ionization field, an air filter, a dual ultraviolet system located within the bio-air sterilization system.

### FIELD OF THE INVENTION

The present invention relates to an air filtration system and, in particular, to an air sterilization unit for removing harmful biological contaminants and particulates from an enclosed area.

### Problem

It is a problem in the field of air filtration systems to remove harmful contaminants and particulates, such as bacteria, viruses and molds, from air within an enclosed area, as well as from the surfaces located within the enclosed area, while also maintaining a safe workplace for individuals.

People spend 75-90% of their time indoors where they are exposed to a growing number of health-threatening indoor pollutants. These pollutants can be categorized into three groups: biological contaminants, such as bacteria, viruses and molds; toxic gases and fumes given off by furniture, carpeting, etc.; and particulates, such as dust and smoke. Approximately half of the major office buildings have contaminated heating, ventilation and air conditioning (HVAC) systems. If not properly maintained, the HVAC systems are a hotbed for the growth of molds and bacteria, regardless of the age of the building. Occupants of these buildings can be expected to suffer from symptoms related to exposure to these health-threatening indoor pollutants. The problem of health-threatening indoor pollutants is exacerbated when the building is a health facility where not only are there a greater number of harmful health-threatening pollutants present, but occupants of the health facility may be more susceptible to maladies caused by these health-threatening pollutants.

A known solution for removing harmful contaminants from ambient air is the use of air purifiers. Air purifiers use a scientifically advanced process that combines the power of genocidal ultraviolet (UV) light, purifying hydroxyl, activated oxygen and photo-ionization for purifying air and sanitizing an area. However, existing air purifiers don't use the multiple approach of pre-ionization of the incoming air, HEPA filtration, and sterilization by use of ultraviolet nm lamps for a more complete solution. Further, most existing air purifiers use small ultraviolet lamps that do not allow adequate time required for sterilization.

Another known solution for removing harmful contaminants from surfaces is the use of chlorine to clean water and surfaces. However, chlorine may leave harmful residuals within the drinking water and chlorine also cannot be readily generated on site. The chlorine must be shipped to the site from manufacturers located a distance away from the point of need. During emergency situations, proper handling of chlorine containers may be impractical.

Because neither of the above-stated solutions is adequate, there is a need for a sterilization system that effectively filters out particulates, safely generates ozone for destroying biological contaminants, and effectively converts the ozone to highly ionized ambient air.

### Solution

The above-described problems are solved by the present bio-air sterilization system that removes harmful contaminants and particulates, such as bacteria, viruses and molds, from air within an enclosed area, as well as from the surfaces located within the enclosed area, while also maintaining a safe workplace for individuals. The bio-air sterilization system uses a compressor to circulate indoor air through an electrostatic filter that generates an pre-ionization field, a mechanical air filter, a dual ultraviolet system located within the bio-air sterilization system.

The pre-ionization field is created by the electrostatic filter when a voltage is applied between the outside shell and the wire mesh of the air filter to pre-ionize the incoming indoor air. The power supply (transformer) applying the charge is fluttered at a high rate (1000 P/s) to create an ionization field for the incoming indoor air. Ions produced by the pre-ionization field attach to particulates in the incoming air, giving them a negative or positive charge so that the particulates may attach to the air filter as the compressor/blower circulates indoor air through the bio-air sterilization system.

The ultraviolet light system consists of two ultraviolet lamps having different radiation wavelengths. The first ultraviolet lamp, operating at 254.2 nanometers (nm) destroys airborne contaminants and particulates in the enclosed area while in operation for a pre-determined period of time. The ions generated by the first ultraviolet lamp attach to airborne particulates and contaminants, giving them a negative or positive charge so that the particles and contaminants attach themselves to nearby surfaces and thereby settle out of the air. At a pre-set time, the first ultraviolet lamp deactivates and the second ultraviolet lamp, operating at 185 nm, is activated and converts oxygen into ozone which is circulated throughout the enclosed area to destroy harmful surface contaminants and particulates. The enclosed area is not safe for occupancy during the time the second ultraviolet lamp is operational at 185 nm. An integrated timer in the programmable logic controller allows for customized control of the second ultraviolet lamp's period of operation. As an additional safety feature, the bio-air sterilization system is fitted with a motion sensor that deactivates the second ultraviolet lamp if someone should enter the enclosed area.

Again at a pre-set time, the second ultraviolet lamp is deactivated and the first ultraviolet lamp is reactivated, operating at 254.2 nm for an appropriate period of time to kill newly introduced airborne contaminants and excess ozone generated by the 185 nm lamp, making the area again safe for occupancy.

An optional, commercial pre-filter can be used for enclosed areas having an excessive amount of dust and/or other heavy particulates in the air.

Alternatively, a corona discharge ozone generator can be substituted for the second ultraviolet lamp to generate higher concentrations of ozone for applications, such as a medical facility, that require higher levels of contaminant destruction.

### BRIEF DESCRIPTION OF THE DRAWINGS

FIG. 1 is a front view (without doors) of the bio-air sterilization system;

FIG. 2 illustrates, in block diagram form, the present bio-air sterilization system;

FIG. 3 illustrates six stages of air treatment performed by the bio-air sterilization system for removing harmful contaminants and particulates from air within an enclosed area, as well as from the surfaces located within the enclosed area; and

FIGS. 4-5 illustrate, in flow diagram form, the operation of the present bio-air sterilization system.

### DETAILED DESCRIPTION

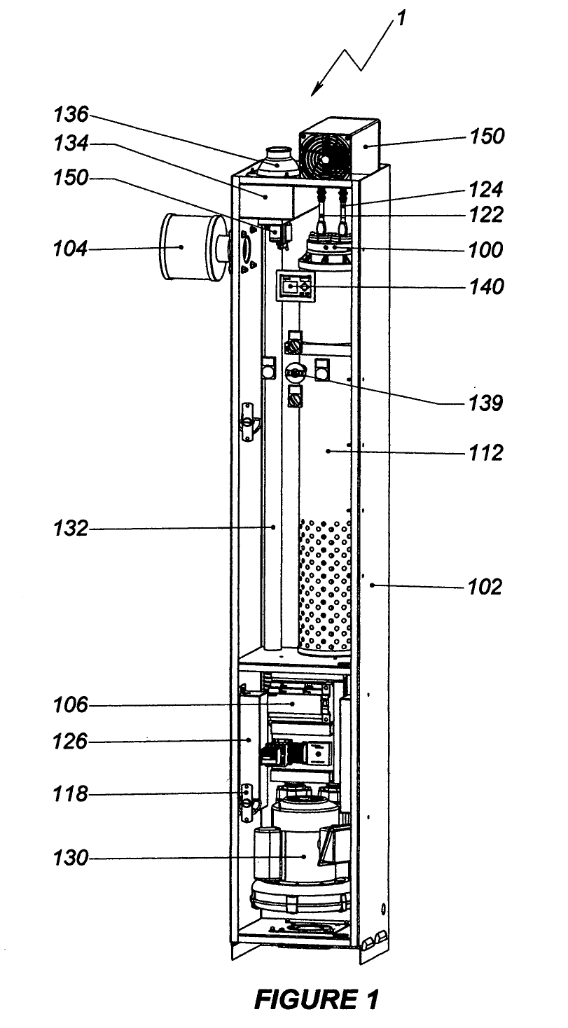

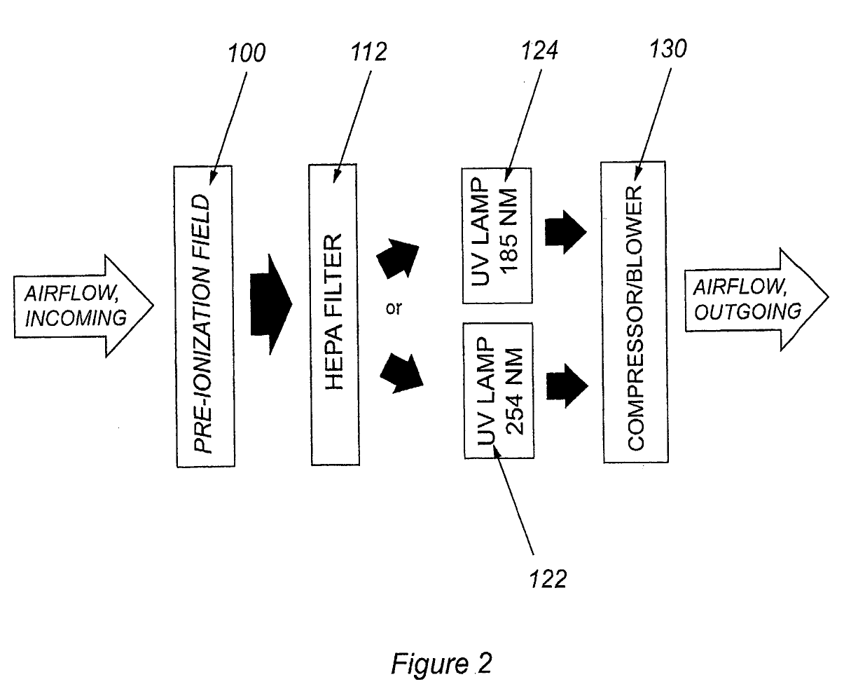

FIG. 1 is a front view (without doors) of the bio-air sterilization system 1, while FIG. 2 illustrates, in block diagram form, the present bio-air sterilization system 1. The bio-air-sterilization system 1 is housed in a cabinet 102 and includes an optional pre-filter 104, a pre-ionization field 100, an air filter unit 112, a dual internal ultraviolet system having a first ultraviolet lamp 122 and a second ultraviolet lamp 124, a transformer 128 to supply the charges to the outer stainless steel shell and the internal mesh shell of the pre-ionization field 100, an air compressor/blower 130 for circulating indoor air through the bio-air sterilization system 1 and a programmable logic controller 140 to allow the user to customize (pre-set) the operation of the bio-air sterilization system 1 for specific needs. An auto mode (on/off) switch 114 allows the user to activate the bio-air sterilization system 1 or deactivate the system. A manual mode switch 116 allows for manual control of the first ultraviolet lamp 122 and the second ultraviolet lamp 124. Door locks 118 provide security for the bio-air sterilization system cabinet 102.

The use of a dual internal ultraviolet system is a photocatalytic oxidation process that combines ultraviolet light of the two different wavelengths, oxygen and the natural humidity found in air, enhanced by the use of high frequency ballast 126 to activate the ultraviolet lamps to create what is known as a hydroxyl radical. The hydroxyl radical is an extremely aggressive oxidizer that neutralizes airborne contaminants and biological and chemical odors within milliseconds, converting them to harmless carbon dioxide and water vapor.

Air Purification/Sterilization Using Ultraviolet/Ozone

The human population is continually exposed to a growing number of health-threatening pollutants that can be categorized into three groups: biological contaminants, toxic gases, and particulates. To deal with these hazards, the present bio-air sterilization system utilizes pre-ionization, filtration, ultraviolet radiation and ozone generation for air purification. First, a voltage supplied by the transformer 128 is applied between the outer perforated stainless steel shell of the pre-ionization field 100 and the internal wire mesh of the filter 112 to pre-ionize the incoming air. Second, the air passes through the high efficiency particulate air (HEPA) filter 112 to which particulates are attached. Third, special wavelengths (254.2 nm) of concentrated ultraviolet light are used to destroy proteins and DNA and in the process, kill harmful airborne contaminants such as bacteria, viruses and molds that depend on these biochemicals for life function. Fourth, photonics ozonation (ultraviolet light at a wavelength of 185 nm) is used to eliminate unpleasant odors, neutralize many harmful gases, and destroy surface contaminants such as bacteria, viruses and fungi. Fifth, purifying hydroxyl, one of the strongest pollutant fighters of all, further destroys surface contaminants. Finally, ions are produced within the dual lamp region and attach themselves to airborne particulates such as dust, pollen, soot, smoke, etc., removing them from the indoor air.

The present bio-air sterilization system is designed for installation in any enclosed area requiring the destruction of bacteria, viruses and odors. For extremely large areas, two or more bio-air sterilization systems may be required and can operate concurrently. The system utilizes a scientifically advanced process that combines the power of genocidal ultraviolet light, purifying hydroxyl, activated oxygen and photo-ionization for the bio-air sterilization system to purify and sanitize indoor air in a defined area. As air passes the radiant field of the special 185 nm ultraviolet lamp 124 it is exposed to a powerful purifying process that destroys harmful contaminants to disinfect surfaces and recessed areas where pathogens may collect. The ultra-purified air is then re-circulated back into the environment and through the radiant field of the first ultraviolet lamp 122 which destroys airborne contaminants which have been newly introduced into the enclosed area and, more importantly, destroys excess ozone produced by the second ultraviolet lamp 124, making the area safe for inhabitants. The air has then become highly ionized, carrying negative ions for removal of particulates from the air and low, safe levels of photonics ozone to seek out and destroy additional hazardous contaminants.

The dual internal ultraviolet system includes operation of a first ultraviolet lamp 122, which operates at a predetermined wavelength of, for example, 254.2 nm, to destroy biological contaminants such as airborne viruses, bacteria, molds, fungi, smoke, and pollution particles. Subsequently, when the second ultraviolet lamp 124, which operates at a predetermined wavelength of, for example, 185 nm, is activated, not only are surface biological contaminants destroyed in the ultraviolet field, but simple oxygen from the indoor air is converted to one of the strongest oxidizers on the planet, ozone. This photocatalytic oxidation process combines ultraviolet light of two different wavelengths, oxygen and the natural humidity found in air, enhanced with a high frequency ballast 126 to create what is known as a hydroxyl radical. The ballast power source for lamp operation 126 utilizes constant wattage circuitry to ensure optimum electronic efficiency and consistent performance. As noted above and as described in more detail below, a corona discharge ozone generator can be substituted for the second ultraviolet lamp to perform the ozone generation function.

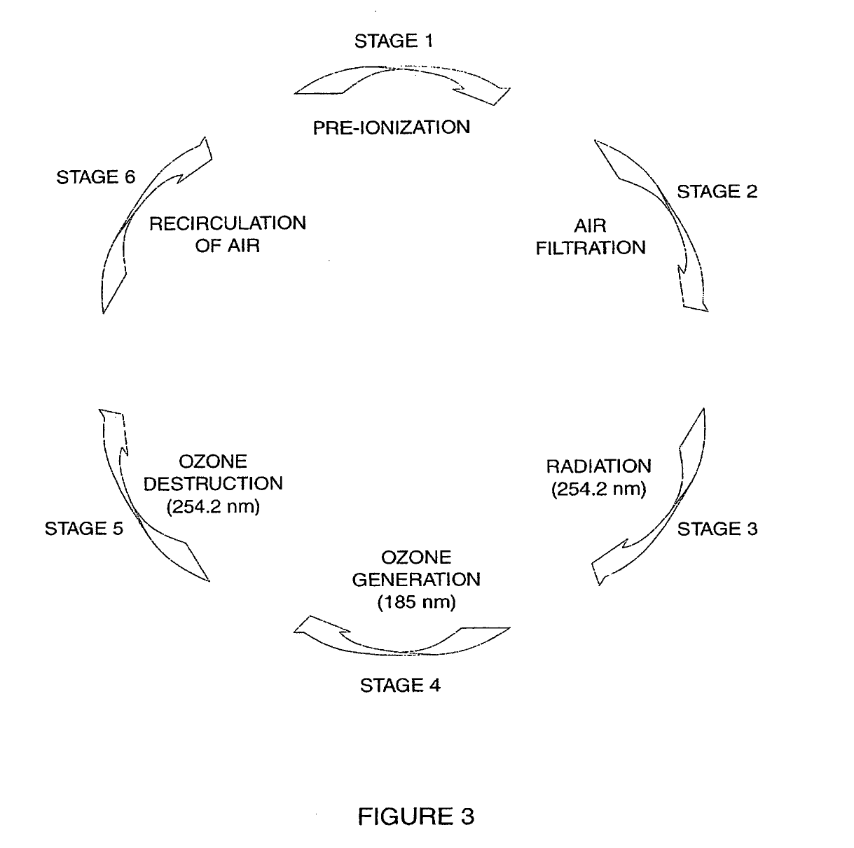

Referring to FIG. 3, the cycle of the present bio-air sterilization includes six stages. In Stage 1, the indoor air is pre-ionized. Ionization through a wire mesh field 100 prior to filtration produces ions that attach to biological contaminants, making the biological contaminants heavier and more likely to be filtered out, as, at Stage 2, the ionized air passes through the HEPA filter 112.

During Stage 3, the filtered air passes through the radiant field of a first ultraviolet lamp 122 where many of the airborne biological contaminants, passing through the radiant field of ultraviolet light at 254.2 nm generated by the first ultraviolet lamp 122, are destroyed. During Stage 4, the ultraviolet light emissions at a wavelength of 185 nm, generated by the second ultraviolet lamp 124, convert the filtered air to ozone as the indoor air circulates through the bio-air sterilization system. Replacing the indoor air with ozone for a period of time destroys surface biological contaminants in addition to any airborne contaminants within the room. At Stage 5, residual ozone and any newly introduced airborne contaminants are destroyed when the first ultraviolet lamp 122 is re-activated and a high level of negative ionization is achieved. At Stage 6 the compressor/blower 130 recirculates the highly ionized air through exit tube 132 and into a mixing chamber 134 (an empty stainless steel box where ozone is mixed by creating turbulence) prior to re-entry into the enclosed area through a directional output nozzle 136 at the top of the unit. The continuous cycle through the bio-air sterilization system then begins again.

Stage One: Pre-ionization

The present bio-air sterilization system contains a built-in pre-ionization field 100. The outside shell of the pre-ionization field 100 is constructed of perforated stainless steel while the outside shell of the interior filter system 112 is covered with a stainless steel wire mesh. A low voltage, 1000 Hz power supply connected to the outside shell applies a + (positive) charge to the outside shell while the same power supply applies a − (negative) charge to the interior stainless steel mesh. This produces an electrical field that charges the airborne contaminants to promote aggregation of particles for easy removal and actually electrocutes a large portion of the airborne pathogens. The power supply is a low voltage, low amperage dc system located within the bio-air sterilization system. The power supply output is then fluttered at over 1,000 times per second to create an ionizing field for the entering indoor air to produce ions.

Stage Two: Filtration

The ions produced in stage one attach to particles in the air giving them a negative or positive charge so that the particles may attach to nearby surfaces within the air filter 112 during the air filtration stage. The air filter 112 meets HEPA specifications to capture particle sizes as small as 0.3 to 0.5 microns. (The smallest particle size visible to the human eye is 40 microns.)

The HEPA filter 112 may be pleated to ensure proper seal and to maximize surface area. The pleated design yields a surface area several thousand times that of a flat filter using the same space requirements. The filter 112 traps a significant amount of the airborne contaminants and particulates such as pollen, molds, and dust that are circulating in the room.

Stage Three: Radiation

The present bio-air sterilization system includes a dual internal ultraviolet system. The daytime operation utilizes a first ultraviolet lamp 122, operating at a wavelength of 254.2 nm, and the nighttime operation utilizes a second ultraviolet lamp 124, operating at a wavelength of 185 nm. Use of a dual internal ultraviolet lamp system is a photocatalytic oxidation process that combines ultraviolet light of two different wavelengths, oxygen, and the natural humidity found in air, enhanced by the use of a high frequency ballast 126 to activate the ultraviolet light generators to create what is known as a hydroxyl radical. The hydroxyl radical is an extremely aggressive oxidizer that neutralizes airborne contaminants and biological and chemical odors within milliseconds, converting them to harmless carbon dioxide and water vapor. Following the first and second stages illustrated in FIG. 3, the third stage exposes the filtered air from the HEPA filter to a radiant field created by a first ultraviolet lamp 122 operated at a wavelength of 254.2 nm to destroy airborne contaminants. This lamp is usually operated during the day when the enclosed area is occupied and disinfection must be restricted to the air passing through the filter system.

Stage Four: Ozone Shock Treatment

Prior to operation of the second ultraviolet lamp 124, the room is vacated. Since most enclosed areas are, presumably, occupied during the daytime, it is assumed the optimum operational hours for the second ultraviolet lamp 124 are at nighttime when most contaminated areas are unoccupied. However, the second ultraviolet lamp 124 can be preset through the programmable logic controller 140 to be operational during any specified period. Although the bio-air sterilization system produces a low concentration of ozone, prolonged exposure could be harmful to human beings. Therefore, the enclosed area should be vacant while ozone is being produced. As an additional precaution, a motion sensor 138 deactivates the second ultraviolet lamp 124 if someone should enter the room while the second ultraviolet lamp 124 is operational. As the filtered air circulates through the radiant field created by operation of the second ultraviolet lamp 124, harmful surface contaminants such as bacteria and viruses are destroyed. The ultraviolet lamps are fired by a ballast 126 having a cycle rate of 35,000 cycles per second compared to the standard cycle rate of 24,000 cycles per second as used on conventional ultraviolet systems. This higher cycle rate greatly enhances the output of the lamp in microwatts per square centimeter.

Operation of the second ultraviolet lamp 124 also converts oxygen from the filtered air into one of the strongest oxidizers on the planet, ozone. The ozone then leaves the bio-air sterilization system and is distributed throughout out the enclosed area. When bacteria and viruses come in contact with the ozone the destruction is immediate. Since the ozone is airborne, it can reach all of the places bacteria and viruses reach, such as bedding, carpet, furniture, floors, walls, the inside of walls, etc. Depending on the contamination in the area to be sterilized and the size of the area, the duration of operation of the second ultraviolet lamp 124 may vary, but it is timed through the programmable logic controller 140 to deactivate well before the area is scheduled for occupancy again.

The optional corona discharge ozone generator makes use of multiple anode/cathode cells. Ozone is created when a gas (ambient air or oxygen) passes through the space between the anode and cathode while a high voltage is applied across the anode and cathode arrangement. As the gas passes through the space between the anode and cathode, the electric field created by the voltage applied to the anode and cathode splits some oxygen molecules, and the released oxygen atoms react with oxygen molecules to form ozone. The corona discharge pack is constructed of a rolled stainless steel, perforated shell. A ceramic dielectric is inserted into the shell and a working gap is established between the ceramic dielectric and the shell to support the corona discharge. The commercial low voltage power is stepped-up to the high voltage necessary to create the corona by a frequency/voltage inverter before delivery to the ceramic core. As it passes through the corona, the oxygen in ambient air becomes excited and its oxygen molecules become disassociated. Some of the oxygen molecules recombine into ozone. The corona discharge ozone generator is designed to produce ozone levels adequate for the required room disinfection. For simplicity of description, the following description refers to a “second ultraviolet lamp” and it is understood that the corona discharge ozone generator can be substituted for the second ultraviolet lamp.

More and more room furnishings and floor coverings are made from synthetic materials. Under the stagnant air conditions that prevail due to the lack of ventilation, decomposition products and solvents leach out of these synthetic materials and fill the indoor space. Much has been said about the “sick building syndrome” which usually refers to institutional buildings—but private homes can have basically the same problems. When odors are inhaled, they cause an increase in the demand for oxygen in the human body, thus depleting the body of oxygen. Ozone reacts rapidly with most odors, oxidizing them into less harmful elements or into harmless carbon dioxide.

Ozone in natural settings is around 0.02 parts per million (ppm), but it can be as high as 0.10 ppm. At this level, ozone is capable of keeping pathogens in check, and yet, at this level, ozone is not harmful to higher life forms such as fish, birds, animals, or man. Ozone is not harmful to nature. Only a prolonged exposure to unnaturally high levels of ozone may lead to discomfort (headache and coughing), signaling one to leave that space and seek better air.

The purpose of the ozone shock treatment is to eliminate contamination from the air and surfaces and provide continuous control over recurring contamination by way of air filtration, ozone, and ultraviolet exposure over a period of time. When ozone comes into contact with bacteria, viruses, fungi and molds, it gives up an atom of oxygen (a free radical) that oxidizes, or destroys, the contaminants. These life forms are anaerobic and cannot live with activated oxygen.

The extent of the odor and contaminant problem determines the size of the ozone generator (bio-air sterilization system) required to sufficiently eliminate the problem of odor, bacteria, and viruses in a reasonable period of time. Once the ideal size of the bio-air sterilization system has been determined, the bio-air sterilization system should be placed in a direction that enhances air circulation in the problem area.

Breathing ozone or drinking ozonated water (at the safe, legal concentrations that are already outlined by the government) are two of the ways to get activated oxygen into a human body. If you have ever taken a drink of water just down stream from a waterfall and felt invigorated, it was because the water had tumbled over the rocks, thinned out and absorbed oxygen/ozone from the air. If it were unsafe to breathe acceptable concentrations of ozone, the military would not use ozone to purify the air in submarines. Oxygen is the first line of defense for the human immune system and is necessary for the removal of every waste product from our bodies.

Stage Five: Ozone Destruction and Ionization

When the second ultraviolet lamp 124 is deactivated and the first ultraviolet lamp 122 is reactivated, the compressor/blower 130 re-circulates the air through the bio-air sterilization system and destroys new airborne contaminants brought into the room and the excess ozone that was generated by the second ultraviolet lamp 124 while the area was unoccupied. The radiant field created by the first ultraviolet lamp 122 not only destroys airborne viruses and bacteria reintroduced by occupants, recirculation of the air through the bio-air sterilization unit also destroys the excess ozone generated by the second ultraviolet lamp 124.

The first ultraviolet lamp 122 also operates as an ionizer, or ion generator. An ionizer disperses negatively and/or positively charged ions into the air. Ions are charged particles in the air that are formed when enough energy acts upon a molecule such as carbon dioxide, oxygen, water or nitrogen to eject an electron from the molecule leaving a positively charged ion. The displaced electron attaches itself to a nearby molecule that then becomes a negatively charged ion.

These ions attach to particles in the air, giving them a negative or positive charge so that the particles may attach to nearby surfaces such as walls or furniture, or attach to one another and settle out of the air. Human exposure to airborne contaminants is considerable; but ionization of the contaminants removes them from the air rendering the air safe to breathe. Circulation of indoor air through the radiant field created by the first ultraviolet lamp 122 destroys airborne contaminants.

Stage Six: Recirculation

A compressor/blower 130 circulates ambient air through the bio-air sterilization system. The compressor/blower 130 operates at approximately 65 cubic feet per minute (CFM) to allow time for the ambient air, following filtration, to reside in the ultraviolet system for destruction of harmful contaminants.

The purified air recirculates through exit tube 132 that extends from the bottom of the bio-air sterilization cabinet 102 through a mixing chamber 134 (an empty stainless steel box where ozone is mixed by turbulence created by “bouncing” of the walls of the box) and exits out into the enclosed area through a directional output nozzle 136 at the top of the bio-air sterilization cabinet 102.

When operated during periods of time when the room is occupied, circulation through the bio-air sterilization system destroys airborne contaminants and filters out particulates that are reintroduced into the environment. The air emitted from the present bio-air sterilization system is highly ionized, free of odor, and will continue to control odor, bacteria and viruses while the system is in operation.

Operation of the Bio-Air Sterilization System

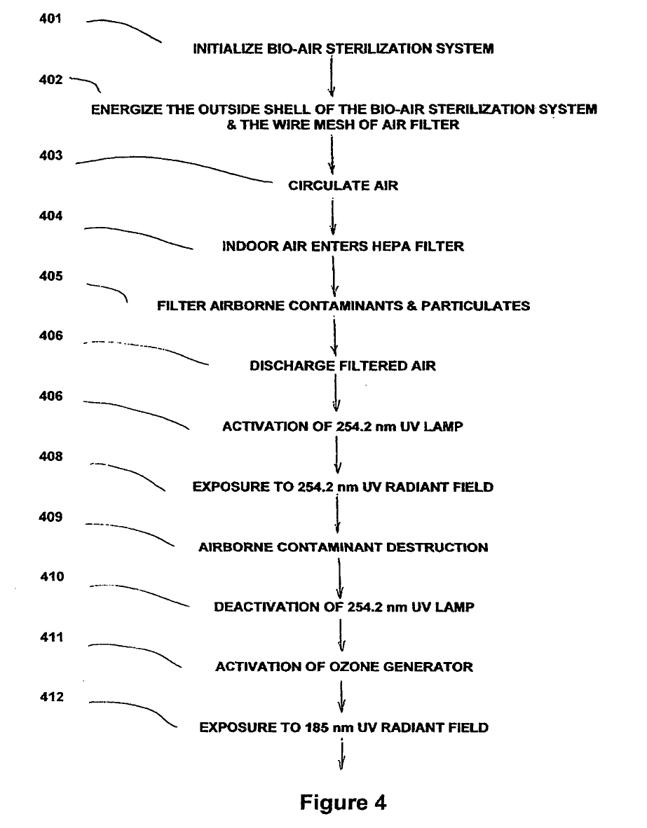

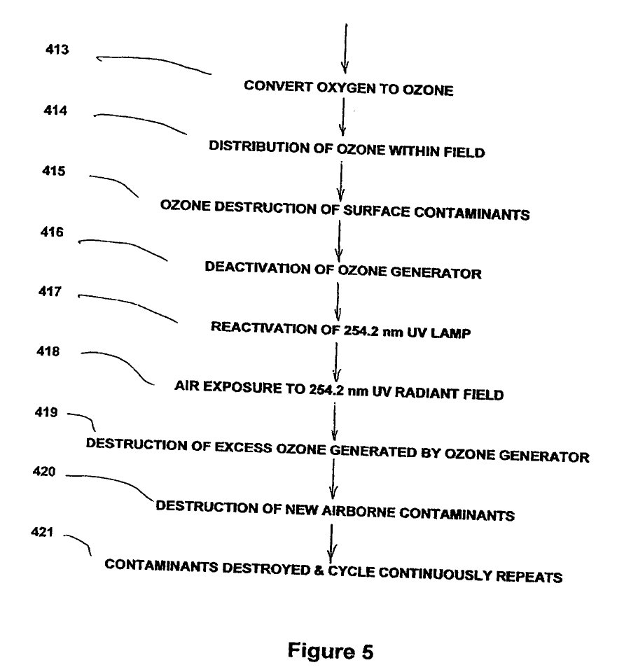

FIGS. 4-5 illustrate, in flow diagram form, the operation of the present bio-air sterilization system. At step 401, the bio-air sterilization system is activated and at step 402 energizes the pre-ionization process by application of a voltage between the outside shell of the unit and the wire mesh shell of the HEPA filter. A compressor/blower, located within the enclosed unit, circulates indoor air through the bio-air sterilization system in step 403 during operation. In step 404 the pre-ionized incoming indoor air is filtered through the HEPA filter where, in step 405, some of the airborne contaminants and particulates are removed. The filtered air is discharged into the ultraviolet system in step 406. At step 407, the first ultraviolet lamp is activated and in step 408 the filtered air from the HEPA filter passes through the radiant field generated by the first ultraviolet lamp. The radiant field destroys airborne contaminants such as bacteria and viruses in step 409. In step 410, the first ultraviolet lamp is deactivated.

The second ultraviolet lamp is activated in step 411 and surface contaminants are exposed to the second ultraviolet radiant field in step 412. Concurrently, in step 413, the filtered air passing through the radiant field is converted to ozone that is expelled from the bio-air sterilization system. In step 414, the ozone is circulated through the enclosed area. The ozone within the enclosed area continues destroying harmful contaminants in step 415 for a period of time during which the method repeats steps 412 through 415. After a pre-determined period of time, the second ultraviolet lamp is deactivated in step 416. Deactivation may be dependent on a prescheduled time by use of an optional timer or an alternative method may be used to determine when to deactivate the second ultraviolet lamp, such as deactivation when an optional ozone detector indicates that the indoor air within the enclosed area reaches preset limits of ozone. As a safety precaution, a motion sensor built into the bio-air sterilization system deactivates the second cycle when anyone enters the room.

After the ozone shock treatment of stage 4 is complete and the bio-air sterilization system has deactivated the second ultraviolet lamp in step 417, the bio-air sterilization system reactivates the first ultraviolet lamp in step 418. When the filtered ozone is exposed to the first ultraviolet radiant field in step 419, excess ozone previously generated by the second ultraviolet lamp is destroyed in step 420. In step 421 the highly ionized air continues to destroy newly introduced contaminants and particulates within the enclosed area. At step 422 the contaminants are destroyed and the cycle continuously repeats.

The extent of the odor and contaminant problem determines the size of the bio-air sterilization system required to provide ozone distribution sufficient to eliminate the problem in a reasonable, enclosed area. The bio-air sterilization system is operated at its highest capacity for as long as it takes to eliminate the problem. In most cases, it takes only a few minutes for a shock treatment. If it is a type of bacteria, virus, or odor (for example, cigarette smoke) that has been absorbed into furniture, walls and/or bedding, the air filtration/sterilization cycle takes only 120-240 minutes with the appropriately-sized ozone generator (bio-air sterilization system).

An ozone monitor and controller may be included to maintain ozone levels in the room at an acceptable level (for example 0.05 ppm as set by the United States Government) during ionization while the room is occupied. Likewise, the present bio-air sterilization system may include an automatic timer located within a control panel to allow the user to set the proper cycle times for the area. In addition, a motion sensor deactivates the second ultraviolet lamp if someone enters the enclosed area while it is operational.

As to alternative embodiments, those skilled in the art will appreciate that the illustrated and described use for the present bio-air sterilization system is for removing particulates and harmful contaminants from indoor air. However, alternative uses may be substituted such as filtration, ionization and ozonation of water. Similarly, while the bio-air sterilization system has been described as having a second ultraviolet lamp for generating ozone in an unoccupied room and a first ultraviolet lamp for destroying contaminants while a room is occupied, ultraviolet lamps operating at wavelengths other than those described may be substituted to perform the same tasks.

1. An air sterilization system for removing biological contaminants and particulates from ambient air and surfaces in an enclosed space, comprising:
compressor means for generating a flow of ambient air through said air sterilization system;
filtering means for filtering said flow of ambient air to remove said harmful biological contaminants and particulates from said air to produce filtered air;
first ultraviolet means for generating ultraviolet radiation at a first wavelength to destroy harmful biological airborne contaminants and particulates in said filtered flow of ambient air;
ozone generator means for converting oxygen from said filtered flow of ambient air into ozone, wherein said ozone destroys said biological contaminants and particulates on said surfaces in said enclosed space; and
control means for activating one of said first ultraviolet means and said second ultraviolet means exclusive of the other of said first ultraviolet means and said second ultraviolet means.

2. The air sterilization system of claim 1 wherein said filtering means comprises:
electrostatic means for pre-ionizing said filtered air to produce an ionized filtered air; and
air filter means for mechanically removing said biological contaminants and particulates from said air.

3. The air sterilization system of claim 1 wherein said control means comprises:
cycle control means for recycling said filtered air through the first ultraviolet lamp means to destroy excess ozone and newly introduced biological contaminants and particulates in said ambient air and converting said ozone to a highly ionized air.

4. The air sterilization system of claim 1 wherein said first ultraviolet means comprises:
first ultraviolet lamp means operable to generate ultraviolet light at a wavelength of approximately 254.2 nanometers.

5. The air sterilization system of claim 1 wherein said ozone generator means comprises:
second ultraviolet lamp means operable to generate ultraviolet light at a wavelength of approximately 185 nanometers.

6. The air sterilization system of claim 1 wherein said ozone generator means comprises:
corona discharge means for generating a high voltage electric field to convert oxygen from said filtered flow of ambient air into ozone, wherein said ozone destroys said biological contaminants and particulates on said surfaces in said enclosed space.

7. The air sterilization system of claim 1 wherein said control means comprises:
a motion sensor to deactivate said second ultraviolet means if someone enters the radiant field; and
an ozone sensor for determining and controlling the ozone level within said enclosed space.

8. A method of operating an air sterilization system for removing biological contaminants and particulates from ambient air and surfaces in an enclosed space, comprising:
generating a flow of ambient air through said air sterilization system;
filtering said flow of ambient air to remove said harmful biological contaminants and particulates from said air to produce filtered air;
generating ultraviolet radiation at a first wavelength using a first ultraviolet lamp to destroy harmful biological airborne contaminants and particulates in said filtered flow of ambient air;
converting oxygen from said filtered flow of ambient air into ozone, wherein said ozone destroys said biological contaminants and particulates on said surfaces in said enclosed space; and
activating one of said first ultraviolet lamp and said second ultraviolet lamp exclusive of the other of said first ultraviolet lamp and said second ultraviolet lamp.

9. The method of operating an air sterilization system of claim 8 wherein said step of filtering comprises:
pre-ionizing said filtered air to produce an ionized filtered air; and
mechanically removing said biological contaminants and particulates from said air.

10. The method of operating an air sterilization system of claim 8 wherein said step of activating comprises:
recycling said filtered air through the first ultraviolet lamp to destroy excess ozone and newly introduced biological contaminants and particulates in said ambient air and converting said ozone to a highly ionized air.

11. The method of operating an air sterilization system of claim 8 wherein said step of generating ultraviolet radiation at a first wavelength comprises:
generating ultraviolet light at a wavelength of approximately 254.2 nanometers.

12. The method of operating an air sterilization system of claim 8 wherein said step of converting oxygen comprises:
generating ultraviolet light at a wavelength of approximately 185 nanometers.

13. The method of operating an air sterilization system of claim 8 wherein said step of converting oxygen comprises:
generating a high voltage electric field to convert oxygen from said filtered flow of ambient air into ozone, wherein said ozone destroys said biological contaminants and particulates on said surfaces in said enclosed space

14. The method of operating an air sterilization system of claim 8
wherein said step of activating comprises:
deactivating said second ultraviolet lamp if someone enters the radiant field; and
determining and controlling the ozone level within said enclosed space.
